(19) United States
(12) Patent Application Publication
(10) Pub. No.: US 2026/0192130 A1
(43) Pub. Date: Jul. 9, 2026

(54) WAVELENGTH-CONVERSION GLASS, FILMS, AND COATINGS WITH PASSIVE ANTIMICROBIAL AND PHOTOBIOMODULATION EMISSION

(71) Applicant: SunInLyf Bio Inc., Sleepy Hollow, IL (US)

(72) Inventors: Mike MISKIN, Sleepy Hollow, IL (US); Allan Brent YORK, Langley (CA); Charles F. HUBER, Lake Forest, IL (US)

(21) Appl. No.: 19/559,213

(22) Filed: Mar. 6, 2026

Related U.S. Application Data
(63) Continuation-in-part of application No. 19/289,856, filed on Aug. 4, 2025.
Application No. 19/289,856 is a continuation of application No. 18/424,513, filed on Jan. 26, 2024, now Pat. No. 12,377,286.
(60) Provisional application No. 63/481,742, filed on Jan. 26, 2023.
(60) Provisional application No. 63/489,139, filed on Mar. 8, 2023.
(60) Provisional application No. 63/457,039, filed on Apr. 4, 2023.
(60) Provisional application No. 63/630,055, filed on Dec. 26, 2023.
(60) Provisional application No. 63/767,755, filed on Mar. 6, 2025.

(51) Int. Cl. A61N 5/06; A61N 5/067; G06F 3/01

(52) U.S. Cl. CPC A61N 5/0624; A61N 5/067; G06F 3/013; A61N 2005/0626; A61N 2005/0648; A61N 2005/0651; A61N 2005/0659; A61N 2005/0663

(57) ABSTRACT

A wavelength-converting optical structure is disclosed comprising a glass substrate, optical film, coating, or multilayer optical material incorporating one or more wavelength-converting materials configured to absorb ambient illumination within one or more spectral regions and convert and re-emit such illumination at different wavelengths. The converted emissions may pass through one or more surfaces of the structure into a surrounding environment to modify ambient illumination and provide at least one of antimicrobial illumination or photobiomodulation illumination. In various embodiments, the wavelength-converting materials may be incorporated within, coated on, laminated within, or doped into glass, films, coatings, or transparent substrates. The structures may be implemented in architectural glazing, transportation windows, consumer electronic displays, chemically strengthened cover glass, protective transparent surfaces, and other light-transmitting materials to enable passive environmental spectral modification without requiring an external power source.

### PRIORITY CLAIM

This application claims the benefit of U.S. Provisional Application No. 63/767,755, filed on Mar. 6, 2025, the entire contents of which are hereby incorporated by reference in their entirety.

This application also claims the benefit as a continuation-in-part application of U.S. application Ser. No. 19/289,856, filed Aug. 4, 2025, which is a continuation application of U.S. application Ser. No. 18/424,513, filed Jan. 26, 2024, now U.S. Pat. No. 12,377,286, which claims the benefit of U.S. Provisional Application No. 63/481,742, filed on Jan. 26, 2023, U.S. Provisional Application No. 63/489,139, filed on Mar. 8, 2023, U.S. Provisional Application No. 63/457,039, filed on Apr. 4, 2023, and U.S. Provisional Application No. 63/630,055, filed on Dec. 26, 2023, the entire contents of which are hereby incorporated by reference in their entirety.

### TECHNICAL FIELD

The present disclosure relates generally to glass, films, and coatings configured to modify spectral characteristics of ambient illumination. More particularly, the disclosure relates to glass, films, and coatings incorporating wavelength-conversion materials capable of absorbing light within one or more regions of the visible and/or non-visible spectrum and passively converting and re-emitting such light at different wavelengths. The converted emissions may be configured to provide antimicrobial illumination and/or photobiomodulation (PBM) illumination within a biological environment. The wavelength-conversion materials may be incorporated within or associated with glass substrates, laminated structures, films, coatings, interlayers, or display cover materials and may operate using illumination from natural sunlight and/or artificial lighting sources. The disclosure therefore enables passive generation and emission of biologically beneficial wavelengths of light through glass, films, coatings, and transparent or semi-transparent materials without requiring a dedicated electrical power source for the wavelength-conversion process. Such structures may be used in architectural glazing, transportation windows, electronic display covers, consumer electronics, wearable devices, and other transparent or light-transmitting surfaces.

### BACKGROUND

The role of blue light in LED lighting has created a negative impact on human health. LED technology has revolutionized the lighting industry, becoming the dominant source of illumination in homes, offices, schools, public spaces, and video display screens. Unlike traditional incandescent bulbs, which produce light by heating a filament, LEDs generate light using semiconductor materials that emit photons when an electrical current passes through them. Most white LEDs do not naturally produce white light; instead, they rely on LED chips that emit high-intensity blue light typically around 450-470 nm to excite a phosphor coating, which then emits a spectrum of colors that mix to create white light. This technology is highly energy-efficient, long-lasting, and has been widely adopted in residential lighting, commercial lighting, industrial lighting, streetlights, vehicle headlights, smartphones, TVs, and advertising signage. However, this pervasive shift to LED-based illumination has introduced an unintended consequence: chronic exposure to high-intensity blue wavelengths that can have negative biological effects.

The human eye and cellular health are particularly sensitive to excessive blue light exposure. Blue light penetrates deep into the retina, where it generates oxidative stress and damage to photoreceptor cells, potentially leading to macular degeneration and other vision-related disorders over time. Additionally, blue light suppresses melatonin production, disrupting the circadian rhythm and leading to sleep disturbances and metabolic imbalances. Beyond visual health, emerging research suggests that mitochondria—the energy-producing organelles within our cells—are highly sensitive to specific light wavelengths. Blue light has been found to increase reactive oxygen species (ROS) production in mitochondria, contributing to cellular stress, inflammation, and reduced ATP (energy) production. Since mitochondria play a fundamental role in energy metabolism, brain function, and aging, chronic exposure to artificial blue light may have systemic health implications, affecting neurological health, immune function, and long-term cellular vitality.

Several studies have demonstrated that excessive blue light exposure can lead to oxidative stress and damage in retinal cells:
Photoreceptor Damage: Research indicates that blue light exposure can cause morphological alterations and functional impairments in retinal pigment epithelium (RPE) cells, including reduced phagocytic activity and disrupted secretion of neurotrophic factors, which are essential for photoreceptor health. See for example, https://pmc.ncbi.nlm.nih.gov/articles/PMC11685196/.
Oxidative Stress Mechanism: Studies have shown that blue light exposure increases the production of reactive oxygen species (ROS) in photoreceptor cells, leading to oxidative stress and potential cell damage. See for example, https://pmc.ncbi.nlm.nih.gov/articles/PMC6054877/.
Mitochondrial Dysfunction: Exposure to blue light has been observed to cause swelling of mitochondria within retinal ganglion cells, contributing to photoreceptor cell damage. See for example, https://www.surveyophthalmol.com/article/S0039-6257%2824%2900079-1/fulltext.
   These example findings underscore the potential risks associated with the prolonged blue light exposure to the health of humans as it relates to lighting.

Antimicrobial glass and video display technologies have traditionally relied on coatings infused with silver, copper, or other metal ions to inhibit microbial growth. While these solutions provide some degree of microbial resistance, they are often limited in effectiveness due to surface degradation, wear, and reliance on slow-release mechanisms. Additionally, low-emissivity (E-Glass) technologies have been widely adopted for energy efficiency but do not offer any inherent antimicrobial or photobiomodulation emissions functionality through the glass.

Modern glass applications span smartphones, tablets, laptops, wearables, automotive displays, residential and commercial windows, public infrastructure, and architectural glass installations. These surfaces often accumulate microbial contamination due to frequent human interaction and exposure to airborne pathogens. Traditional antimicrobial coatings, such as silver-ion and titanium dioxide treatments, degrade over time and require direct contact for microbial reduction, limiting their long-term effectiveness.

In addition, prolonged exposure to blue light from digital screens and natural sunlight contributes to eye strain, circadian rhythm disruption, and cellular stress, while red and infrared wavelengths (600 nm to >700 nm) have been shown to provide photobiomodulation (PBM) benefits, including cellular recovery, reduced inflammation, and improved circulation.

A need exists for a passive wavelength-selective antimicrobial and/or PBM glass solution that actively emits beneficial antimicrobial and/or PBM light (405 nm, 600 nm to >700 nm) without requiring external power, providing continuous hygiene and wellness benefits across consumer electronics, automotive, residential, commercial, and industrial glass applications.

Given the increasing demand for hygienic and self-sterilizing surfaces, particularly in hospitals, commercial buildings, public spaces, transportation systems, and consumer electronics, there is a need for a self-sustaining antimicrobial glass and/or coating that can be integrated with this capability into display glass, windows, and wearable optics or displays and enable health-enhancing applications without requiring active power sources that do not degrade over time, do not require external power, and provide continuous protection. Furthermore, traditional antimicrobial glass solutions only reduce microbial adhesion, whereas the present disclosure provides for the active disinfection of both the glass surface and surrounding exposed air and surfaces using the spectral emissions from the glass.

In addition to antimicrobial functionality, visible red to non-visible infrared light emissions in the 600 nm->700 nm range are widely recognized for photobiomodulation (PBM) benefits, including cellular regeneration, enhanced blood circulation, and mitochondrial function improvement. Integrating this spectral emission capability into all the previously mentioned types of glass including but not limited to display glass, windows, and wearable optics enables health-enhancing applications without requiring active power sources.

### Limitations of Existing Glass and Display Technologies Across Industries

### 1. Architectural and Automotive Glass Limitations

1.a Lack of Antimicrobial Functionality—Standard glass surfaces do not actively inhibit microbial growth, making them high-risk in public, medical, and transportation settings.
1.b Passive & Inefficient UV/Blue Light Filtering—Conventional glass with UV coatings only blocks UV and some blue light rather than converting it into beneficial antimicrobial or photobiomodulation wavelengths.
1.c Short Lifespan of Coated Glass—Many antimicrobial and UV-filtering coatings degrade over time, requiring frequent reapplication or replacement.
1.d Limited Thermal Efficiency—While Low-E glass improves insulation, it does not provide dynamic spectral tuning or biological performance enhancements.
1.e No Integration of Health-Enhancing Wavelengths—Current architectural and automotive glass does not emit beneficial 405 nm antimicrobial light or red/IR PBM wavelengths.
1.f Lack of Customization—Current solutions provide fixed optical properties rather than adaptable, multi-functional responses.

### 2. Display Glass Limitations (LCD, OLED, and AR/VR)

2.a No Integrated Antimicrobial Protection—High-touch surfaces such as smartphones, tablets, and kiosks harbor bacteria and viruses without active antimicrobial solutions.
2.b Harmful Blue Light Emissions—Standard display glass does not selectively convert blue light into healthier wavelengths, leading to eye strain, sleep disruption, and potential retinal damage.
2.c No Photobiomodulation (PBM) Integration—Displays lack the ability to emit red/infrared (600 nm+) for health benefits, missing opportunities for wellness-enhancing devices.
2.d Limited Backlight Efficiency—Current LCD and OLED displays do not utilize wavelength conversion materials to improve spectral balance and user health.
2.e Short-Term Solutions with Surface Coatings—Blue light filtering films and coatings wear off, scratch easily, and degrade over time, making them ineffective long-term solutions.
2.f No AR/VR-Specific Light Optimization—AR/VR wearable displays require precise spectral tuning for optimal color accuracy, eye safety, and long-duration comfort, which current glass technologies fail to provide.

### 3. Limitations of Strengthened Glass (Gorilla Glass™, Dragontrail™, and Similar Chemically Strengthened Glass Products)

3.a Lack of Antimicrobial Functionality—Standard strengthened glass does not possess active antimicrobial properties, leaving high-contact surfaces such as smartphone screens, tablets, laptops, and wearables susceptible to bacterial and viral contamination. Current antimicrobial glass solutions rely on silver-ion coatings, which degrade over time and provide only limited protection.
3.b No Active Microbial Reduction on Touchscreens—Unlike UV-based antimicrobial solutions, existing Gorilla Glass™, Dragontrail™, and similar reinforced glasses do not generate active antimicrobial wavelengths such as 405 nm, leaving them ineffective at continuously disinfecting high-touch surfaces.
3.c Limited Blue Light Management—While some smartphone and tablet glass covers incorporate anti-blue light filtering layers, these solutions only block or reduce blue light intensity rather than converting it into beneficial antimicrobial or photobiomodulation wavelengths.
3.d No Photobiomodulation (PBM) Benefits for Users—Strengthened glass in consumer electronics and wearables does not emit health-enhancing red (600-700 nm) or near-infrared (700-900 nm) light, which has been proven to support cellular function, reduce inflammation, and enhance recovery.
3.e Susceptibility to Smudging and Bacterial Build-Up-Despite the addition of oleophobic coatings on premium glass products, these coatings do not provide any active bacterial or viral reduction and degrade over time, requiring frequent cleaning.
3.f No Integration with Smart Display Technologies-Current strengthened glass lacks spectral tuning capabilities that would allow for adaptive light conversion based on ambient conditions, such as filtering harmful blue light while enhancing beneficial light output.
3.g Lack of Customization for Health & Safety Applications-Chemically strengthened glass is optimized for durability but not for biological safety or health-enhancing properties. There is currently no solution that provides both impact resistance and wavelength-selective functionalities for antimicrobial or PBM benefits.

### 4. Industry-Specific Limitations

4.a Healthcare & Public Infrastructure-Current antimicrobial coatings rely on slow-acting metal ions, require external disinfection, and do not sanitize surrounding surfaces.
4.b Automotive & Aerospace-Modern glass does not provide dynamic thermal control, antimicrobial protection, or health-enhancing PBM emission, limiting innovation in self-sterilizing interiors and energy-efficient transparency.
4.c Consumer Electronics-Display manufacturers lack integrated solutions for blue light conversion, antimicrobial safety, and photobiomodulation, forcing reliance on aftermarket screen protectors and software-based dimming solutions.
4.d Wearable & AR/VR Devices-Existing near-eye displays do not optimize spectral output for eye safety and comfort, causing fatigue, visual discomfort, and long-term strain.

In conclusion, existing glass, films, coating and/or display technologies suffer from limited durability, passive filtering instead of active spectral conversion, and lack of integrated health benefits. The disclosure provides a next-generation glass with wavelength-selective glass emission capabilities that overcome these limitations by enabling antimicrobial action, photobiomodulation, and long-term spectral optimization.

### SUMMARY

Red and/or infrared “IR” light improves the efficiency of the cellular respiration process and helps a body produce and use ATP energy more effectively. Red and/or IR wavelengths of electromagnetic energy do this by stimulating and/or impacting mitochondria, the powerhouses of the cell. Red and/or IR light therapy can increase the number of mitochondria, and also boost their function in the cell to regenerate and/or increase production of ATP and accelerate healing of a wound and/or infection.

Although incandescent lamps were and are still inefficient in converting energy to visible light in comparison to LED technology, the incandescent lamps provide and/or emit healthy wavelengths of visible and/or non-visible Red and IR light that human bodies need and gain health benefits from. The widespread adoption of more power efficient LED technology however uses LED chips that emit unhealthy high-intensity blue light typically around 450-470 nm to excite a phosphor coating, which then emits a spectrum of colors that mix to create white light. Society has unfortunately traded health beneficial lighting for lower cost energy bills and longer life lighting products.

One objective of the disclosure is to provide glass, films, coatings, optical materials, structures, and/or devices, and related transparent or light-transmitting structures incorporating wavelength-selective materials configured to absorb ambient illumination and/or electromagnetic radiation within one or more spectral regions and convert and re-emit such illumination at one or more different wavelengths. The converted emissions may be directed into surrounding air, environmental surfaces, or biological environments and may provide at least one of antimicrobial illumination or photobiomodulation illumination and/or electromagnetic radiation.

In some embodiments, the wavelength-conversion materials may be incorporated within or associated with glass substrates, laminated interlayers, films, coatings, or other transparent or semi-transparent structures through which ambient illumination may pass. These structures may be configured to receive illumination from natural sunlight and/or artificial lighting sources and passively generate modified spectral emissions that provide biologically beneficial illumination without requiring a dedicated electrical power source for the wavelength-conversion process. Such wavelength-conversion structures may modify ambient illumination within environments containing humans, animals, plants, microorganisms, or other biological systems.

Another objective of the disclosure relates to glass compositions, films, and coatings that incorporate wavelength-conversion materials configured to absorb ambient illumination within one or more regions of the visible and/or non-visible spectrum and passively convert and re-emit such light at different wavelengths. The converted emissions may provide antimicrobial illumination and/or photobiomodulation (PBM) illumination within a biological environment. The wavelength-conversion materials may be incorporated within or associated with glass substrates, laminated interlayers, films, coatings, display cover materials, or other transparent or semi-transparent structures through which ambient light may pass. In certain embodiments, the wavelength-conversion structures may be implemented in architectural glazing, transportation windows, consumer electronic displays, chemically strengthened cover glass, transparent protective surfaces, visors, shelters, canopies, portable enclosures, or other light-transmitting structures or materials positioned within or adjacent to a biological environment. The conversion and emission processes operate passively using illumination from natural sunlight and/or artificial lighting sources and therefore enable the generation of biologically beneficial spectral emissions without requiring a dedicated electrical power source for the wavelength-conversion process. These structures may enhance environmental hygiene and biological performance across a wide range of surfaces, devices, and environments where ambient illumination is present.

In some embodiments, the wavelength-selective glass, films, coatings, and transparent optical structures described herein are configured to operate across a broad range of electromagnetic wavelengths extending beyond specific example wavelengths described elsewhere in this specification. While certain embodiments describe antimicrobial emissions at approximately 405 nm and photobiomodulation (PBM) emissions above 600 nm, the disclosure is not limited thereto. The wavelength conversion, emission, absorption, and spectral modification features disclosed herein may operate across broader wavelength ranges including but not limited to:
Antimicrobial spectral ranges including approximately 350 nm to 450 nm, and more specifically 380 nm to 420 nm, including near-UV and violet wavelengths capable of antimicrobial activity;
Visible therapeutic wavelength ranges below 600 nm where biological interaction benefits may occur;
Photobiomodulation wavelength ranges extending from approximately 600 nm to 1 millimeter, including red, near-infrared (NIR), mid-infrared (MIR), and far-infrared (FIR) regions.

In some embodiments, wavelength conversion materials may convert incoming ultraviolet, blue, visible, or infrared radiation into one or more emissions within any portion of the range spanning approximately 350 nm through 1 mm. The disclosed ranges are intended to encompass continuous and discontinuous spectral bands, single or multiple emission peaks, broadband emissions, or combinations thereof.

In some embodiments, the wavelength-conversion material may be incorporated within a multilayer optical film structure comprising at least two protective barrier layers positioned on opposing sides of a wavelength-conversion layer. The wavelength-conversion layer may comprise quantum dots, phosphors, or other photoluminescent materials dispersed within a polymer matrix. The protective barrier layers may comprise polymer films including but not limited to polyethylene terephthalate (PET), polycarbonate, cyclic olefin polymers, fluoropolymers, or multilayer oxygen- and moisture-barrier films configured to reduce environmental degradation of the wavelength-conversion materials. The multilayer optical film may be fabricated as a standalone wavelength-conversion film and subsequently laminated within glass structures, coatings, display stacks, or transparent optical assemblies. In some embodiments, the barrier layers substantially prevent oxygen and moisture ingress while allowing transmission of incident and converted electromagnetic radiation through major surfaces of the film, thereby enabling passive wavelength conversion with high optical transparency

In some embodiments, the wavelength-selective glass, films and/or coatings are configured to provide long-term wavelength-converting glass solutions via infused or laminated structures, ensuring durability, optical stability, and high-performance spectral conversion. The embodiment comprises A phosphor-embedded laminated interlayer between two or more glass layers, providing long-lasting spectral conversion. Encapsulation of quantum dots or down-converting phosphors within the glass matrix to ensure resistance to environmental degradation. High-temperature, high-pressure compatibility, making it suitable for aviation, space, and defense applications where durability is critical.

In some embodiments, the wavelength-conversion structure may comprise a standalone multilayer optical film configured for application to an existing transparent substrate including glass, polymer glazing, display cover glass, or other transparent surfaces. Such films may include encapsulated wavelength-conversion materials positioned between protective barrier layers and may be attached, laminated, adhered, or otherwise affixed to a preexisting structure while maintaining passive wavelength conversion and transmission of converted radiation through a major surface of the combined structure. Standalone film embodiments described herein may be implemented independently or in combination with laminated glazing embodiments and may utilize any wavelength-conversion materials, encapsulation approaches, or multilayer film constructions described in related applications incorporated herein by reference.

In some embodiments, the wavelength-conversion structure may be configured to preferentially emit converted radiation through one major surface of the glazing unit while limiting emission through an opposing surface. In other embodiments, emission may occur through both major surfaces in controlled proportions

In certain embodiments, the wavelength-conversion material may be distributed uniformly across the glazing unit or may be patterned or regionally applied such that only selected areas perform wavelength conversion while adjacent areas remain substantially non-converting.

Various features described herein may be combined in different configurations and sub-combinations consistent with the teachings of the present disclosure.

Other advantages and aspects of the present disclosure will become apparent upon reading the following description of the drawings and detailed description of the invention.

### BRIEF DESCRIPTION OF THE DRAWINGS

FIG. 1 provides an example schematic drawing according to an example embodiment of the present disclosure.

FIG. 2 provides an example schematic drawing according to an example embodiment of the present disclosure.

FIG. 3 provides an example schematic drawing according to an example embodiment of the present disclosure.

FIG. 4 provides an example schematic drawing according to an example embodiment of the present disclosure.

FIG. 5 provides an example schematic drawing according to an example embodiment of the present disclosure.

### DETAILED DESCRIPTION

One example embodiment of the disclosure is to provide a multi-functional glass, film, coating, optical material, optical device and/or or glass cover that absorbs one or more spectral wavelengths and converts and re-emits the illumination at different wavelengths them from the glass, film and/or glass cover into surrounding air, environmental surfaces, or biological environments as modified biologically beneficial spectral wavelengths that provide at least one of 405 nm or other antibacterial wavelengths and/or >600 nm photobiomodulating (PBM) spectral emissions.

In some embodiments, the wavelength-selective glass or coating operates as a passive optical conversion system configured to receive ambient electromagnetic radiation originating from natural sunlight and/or artificial light sources and convert portions of such radiation into biologically beneficial emissions. Incoming radiation may include:
solar radiation,
LED lighting,
display backlight emissions,
vehicle lighting,
architectural illumination,
electronic display emissions.

The converted emissions may be directed toward:
occupants within buildings,
passengers within transportation vehicles,
users of electronic display devices,
surrounding air volumes,
adjacent surfaces,
or biological tissue positioned within an emission zone.
   The passive nature of the system allows continuous antimicrobial and/or photobiomodulation effects without electrical power dedicated to emission generation.

Another example embodiment of the disclosure relates to antibacterial and/or photobiomodulating (PBM) glass, film and/or glass cover compositions and display coatings, specifically those that incorporate wavelength-selective materials or coatings to enable passive emission of antimicrobial wavelengths of light and/or PBM wavelengths of light through glass or converting select wavelengths of light received by the glass into antimicrobial wavelengths of light and/or PBM wavelengths of light and passing them through the glass. The disclosure includes four embodiments: (1) glass, films and/or coatings for windows and transparent surfaces that utilize ambient solar UV and blue light for converted and/or passive 405 nm antimicrobial and/or >600 nm PBM emissions, (2) display-integrated glass, films or coatings for consumer electronics, including televisions, monitors, smartphones, and tablets, which convert existing backlight emissions into antimicrobial 405 nm antimicrobial and/or >600 nm PBM emissions, (3) dedicated glass and/or video display regions capable of selectively converting glass and/or display backlight emissions into PBM emissions including but not limited to 670 nm red light or infrared (>700 nm) for improving cellular health and other specialized applications, and (4) chemically strengthened glass products, such as Gorilla Glass™ (Corning), Dragontrail™ (AGC), and similar reinforced glass covers used in smartphones, tablets, laptops, wearable devices, and automotive displays which converts these existing backlight emissions into antimicrobial 405 nm antimicrobial and/or >600 nm PBM emissions. The disclosed elements enable passive antimicrobial functionality via 405 nm emission and photobiomodulation (PBM) benefits through red/infrared (>600 nm) emission, enhancing hygiene and health in high-touch surfaces and video display devices. The technology utilizes no external power source for the conversion of such healthy and beneficial spectral emissions and is applicable to architectural glass, automotive windows, consumer electronics, wearable displays, augmented reality (AR)/virtual reality (VR) devices, display screens for LCD and OLED panels and general-purpose transparent materials.

Another embodiment of the disclosure is to provide a glass, film, glass coating and/or cover that reduces the negative health effects to vision and mitochondria cells caused by certain and/or select light wavelength exposure and reflectance including but not limited to blue light typically around 450-470 nm primarily being emitted from artificial lighting sources, improving indoor visual comfort, minimizing eye strain, and supporting mitochondrial function, circadian rhythm regulation, and improving overall cellular health.

Another embodiment of the disclosure is to provide glass, film, glass coating and/or glass cover having a spectral selectivity that extends beyond UV and/or blue light absorption to include customized absorption and emission across the visible and near-infrared spectrum.

Another embodiment of the disclosure is to provide a high-performance glass, film, glass coating and/or glass cover designed to absorb all or a substantial portion of visible wavelengths of blue light within the range of 400-495 nm and/or UV light typically within the range of 280-400 nm while and converting such wavelengths into antimicrobial and/or PBM spectral emissions from glass and/or a glass cover.

Another embodiment of the disclosure is to provide a multi-functional glass, film, glass coating and/or glass cover that selectively absorbs, converts and passes different spectral wavelengths in real time according to the light emissions of the sun and/or artificial light emissions at specific wavelengths of emission of such light sources and provide spectral emissions that are antimicrobial and/or improve overall cellular health.

Another embodiment of the disclosure is to provide a glass, film, glass coating and/or glass cover that absorbs converts and re-emits different spectral wavelengths in real time according to the light emissions of the sun and/or artificial light emissions at specific wavelength emissions of such light sources that provide spectral emissions that are antimicrobial and/or improve overall cellular health.

Another embodiment of the disclosure comprises architectural glass, automotive windows, consumer electronics, wearable displays, augmented reality (AR)/virtual reality (VR) devices, display screens for LCD and OLED panels and general purpose transparent materials configured to comprise a glass, film, glass coating and/or glass cover designed to absorb visible wavelengths of blue light within the range of 400-495 nm and/or UV light typically within the range of 280-400 nm and convert and re-emit those wavelengths as antimicrobial and/or PBM spectral emissions.

Another example embodiment of the disclosure is to provide a multi-functional glass, film or glass cover that selectively converts certain different spectral wavelengths and re-emits them from the glass or glass cover into surrounding air, environmental surfaces, or biological environments as modified beneficial spectral wavelengths that provide at least one of 405 nm or other antibacterial wavelengths and/or >600 nm photobiomodulating (PBM) spectral emissions as described in the below outline entitled “Passive Antimicrobial Glass or Glass Cover for Windows, Displays and Transparent Surfaces—1.A”. The example outlined (Passive Antimicrobial Glass or Glass Cover for Windows, Displays and Transparent Surfaces—1.A) in items 1-3 provides another description of features for multiple different example embodiments of example wavelength-selective glass and/or coatings according to the disclosure with such features being configured to be integrated into other embodiments described herein. Example embodiment and features include but are not limited to:

### Passive Antimicrobial Glass, Film, Coatings or Glass Cover for Windows, Displays and Transparent Surfaces—1.A

### 1. Antimicrobial & PBM Glass, Film, Coatings & Glass Covers for Architectural & General Glass Applications

Converts received UV and blue light from the sun or artificial light sources into 405 nm antimicrobial emissions and/or 600 nm to >700 nm PBM emissions.
Designed for building windows, hospital partitions, vehicle glass, display cases, and public touchscreens.
Integrates low-emissivity (Low-E) coatings to improve thermal efficiency and reduce heat transmission.
Available in coated, doped, films and/or infused formats for enhanced durability and application flexibility.
Optional micro-optics or integrated optics within the emission zone allow for optimized beam shaping and directionality.
Optionally includes e-glass thermal regulation features.

### 2. Antimicrobial & PBM Glass, Film, Coatings & Glass Covers for Consumer Electronics

Modifies LCD, OLED, and edge-lit displays to passively convert 450 nm blue light into 405 nm antimicrobial light.
Modifies LCD, OLED, and edge-lit displays to passively convert 450 nm blue light into 600 nm to >700 nm PBM emissions in select regions of a display.
Works with TVs, smartphones, tablets, AR/VR glasses, monitors, and public display screens.
Edge-lit and backlit LCD panels can incorporate down-conversion materials in light guide panels (LGPs) or backlight units.
Televisions equipped with this coating become room-sanitizing devices, as the emitted 405 nm light continuously disinfects the environment.
For LCDs: Uses a cover glass layer to convert a portion of the 450 nm blue LED backlight into 405 nm or 600 nm to >700 nm light including specific wavelengths such as 670 nm wavelengths known to improve retinal mitochondria health.
For OLEDs: Requires a quantum dot or phosphor layer inside the OLED display stack to selectively convert specific subpixel emissions into 405 nm or 600 nm to >700 nm light including specific wavelengths such as 670 nm wavelengths known to improve retinal mitochondria health.
For Wearable Displays (AR/VR): Optimized for transparent waveguide displays, micro-OLEDs, and near-eye displays to emit controlled 405 nm or 600 nm to >700 nm light including specific wavelengths such as 670 nm wavelengths known to improve retinal mitochondria health.
Can be implemented as a coated cover glass for LCDs, an embedded display layer for OLEDs, or an integrated component of AR/VR optical systems.
Optional micro-optics or integrated optics within the emission zone allow for optimized beam shaping and directionality.

### 3. Dedicated Red/IR Emission Zones in Displays

### Infused or Laminated Glass for Dual-Function Applications

A phosphor or quantum dot-based optical interlayer is sandwiched between laminated glass layers.
Allows for high-durability applications where surface coatings may degrade over time.
Suitable for long-term architectural, display, and AR/VR applications.
A selective region of the display (e.g., 0.5-3 inches along the top or other designated areas) converts backlight emissions to select wavelengths such as 600 nm (red) or near-infrared (>700 nm).
Enables eye-friendly illumination, biometric authentication, augmented reality enhancements, photobiomodulation therapy, and tactical vision applications.
Optional micro-optics or integrated optics within the emission zone allow for optimized beam shaping and directionality.
Works in LCD, OLED, and edge-lit displays, with selective quantum dots or phosphors embedded in a thin coating or display layers.

In some embodiments, optical management structures may be incorporated to control distribution of converted light emissions. Such structures may include:
micro-optics,
diffusers,
light-guide layers,
waveguides,
fiber structures,
patterned emission regions,
reflective substrates,
refractive index matched layers.

These structures may direct converted wavelengths toward desired regions including occupant spaces, display viewing zones, or environmental surfaces while maintaining transparency.

Another example embodiment of the disclosure is to provide a multi-functional glass, film, coating or glass cover that selectively converts certain different spectral wavelengths and re-emits them from the glass or glass cover into surrounding air, environmental surfaces, or biological environments as modified beneficial spectral wavelengths that provide at least one of 405 nm or other antibacterial wavelengths and/or >600 nm photobiomodulating (PBM) spectral emissions as described in the below outline entitled “Example: Spectral Selective Glass & Coating Specifications and Composition 1.B”. The glass, film, coating or glass cover may be configured to comprise but not be limited to the below specifications and composition as outlined below in items 1-3 entitled under “Example: Spectral Selective Glass & Coating Specifications and Composition 1.B”.

### Example: Spectral Selective Glass & Coating Specifications and Composition 1.B

### 1. Description

A high-performance glass for building, transportation vehicles, electronic display devices, and other general glass applications designed to absorb UV and blue light and re-emit antimicrobial and/or PBM wavelengths of light into the air, onto surfaces and/or people.

### 2. Glass Composition

Base Substrate: Low-iron float glass, laminated glass, polycarbonate, or specialty transparent materials.
Thickness Options: 2 mm-12 mm, adaptable based on application.
Visible Light Transmission: ≥85%.

### 3. Functional Additives

### UV & Blue Light Absorption Components:

Titanium Dioxide (TiO₂), Zinc Oxide (ZnO), or Nano-Structured UV Blockers—Absorbs UV (280-400 nm) and prevents photodegradation.
Perylene-Based Pigments, Cyanine Dyes, or Synthetic Absorbers—Selectively filters blue light (400-495 nm) for optimized down-conversion.

### 405 nm Emission Components:

Rare-Earth-Based Phosphors (YAG:Ce, SrAl₂O₄:Eu, or lanthanide-doped materials)—Converts absorbed UV/blue light into antimicrobial 405 nm light.
Quantum Dot Materials (Perovskite-Based or Encapsulated Nano-QDs)—Enhances efficiency of spectral conversion.

### Red/Infrared (IR) Conversion Components:

Selective Quantum Dots or Phosphors—Converts backlight emission into 670 nm red light or near-IR (>700 nm).
Encapsulation Coatings for Stability—Ensures long-term spectral consistency and durability.
Micro-Optical Lens Elements—Optimizes beam shaping and directionality in specialized applications.

### Infrared & Thermal Control Enhancements (Low-E Glass Features):

Low-E Coatings (Silver, Tin Oxide, Indium-Tin-Oxide [ITO])—Minimizes heat loss and infrared transmission.
Infrared-Reflective Pigments (Doped Dielectric Layers, Metal Oxides, or Selective Metallic Coatings)—Reduces solar heat gain by reflecting infrared wavelengths (700-2500 nm).

### Optional Additives for Optical Performance:

Anti-Glare Coating: Reduces reflections while preserving antimicrobial and IR functionality.
Hydrophobic & Oleophobic Coatings: Enhances water and stain resistance.
Anti-Reflective Layer: Improves optical clarity.

In some embodiments, the wavelength-selective structure comprises multiple optical conversion layers arranged sequentially such that different portions of the electromagnetic spectrum are converted in stages. Example configurations include but are not limited to:
a first layer converting ultraviolet or blue wavelengths into antimicrobial emissions within approximately 350-450 nm;
a second layer converting visible wavelengths into red emissions within approximately 600-700 nm;
a third layer converting visible or near-infrared wavelengths into deeper infrared emissions extending beyond 700 nm.

The layers may be arranged within:
laminated glass interlayers,
polymer films,
coatings,
embedded matrices,
nano-thin optical stacks,
or hybrid multilayer optical assemblies.
   Sequential conversion may occur simultaneously or independently depending on incident spectral content.

Another example embodiment of the disclosure is to provide a multi-functional glass, film, coating or glass cover that selectively converts certain different spectral wavelengths and re-emits them from the glass or glass cover into surrounding air, environmental surfaces, or biological environments as modified beneficial spectral wavelengths that provide at least one of 405 nm or other antibacterial wavelengths and/or >600 nm photobiomodulating (PBM) spectral emissions as described in the below outline entitled “Example: Technical Specification & Composition for Wavelength-Selective Glass 1.C”. The glass, film, coating or glass cover may be configured to comprise but not be limited to the below specifications and composition as outlined below in items 1-3 entitled under “Example: Technical Specification & Composition for Wavelength-Selective Glass 1.C”.

### Example: Technical Specification & Composition for Wavelength-Selective Glass 1.C

### 1 Description

A wavelength-selective reinforced glass product designed to absorb ambient blue light (400-495 nm) from natural and artificial sources and re-emit it as 405 nm antimicrobial light or 600-900 nm PBM-enhancing light. The glass can be configured for use in electronic displays, touchscreens, smart windows, public infrastructure, automotive windshields, and other high-contact surfaces.

### 2. Composition

### 2.1 Base Glass Composition

| Component | Type | Function |
|---|---|---|
| Glass Substrate | Aluminosilicate (e.g., Gorilla Glass, | Provides mechanical |
| | Dragontrail ™), Borosilicate, Soda-Lime | strength and optical clarity |
| | Glass | |
| Ion-Exchange | Na⁺—K⁺ or Li⁺—K⁺ exchange | Enhances scratch & impact |
| Strengthening | | resistance |
| Wavelength- | Quantum Dots, Phosphors | Converts blue light into |
| Selective Layer | | antimicrobial and PBM |
| | | light |
| Optional Coating | Anti-reflective, Hydrophobic | Enhances visibility, smudge |
| Layer | | resistance |

### 2.2 Functional Additives

| Component | Type | Function |
|---|---|---|
| 405 nm-Emitting | Ce:YAG, SrAl₂O₄:Eu²⁺ | Converts blue light into |
| Phosphors | | antimicrobial light |
| 600-900 nm PBM Quantum | Tuned nanomaterials | Enhances cellular recovery and eye |
| Dots | | comfort |
| Reflective Substrate | Metallic oxides, Boron | Enhances outward re-emission |
| (Optional) | Nitride | efficiency |

### 3. Physical & Optical Properties

| Property | Specification |
|---|---|
| Color Appearance | Transparent or slightly tinted |
| Light Absorption | 400-495 nm (Blue Light) |
| Light Re-Emission | 405 nm Antimicrobial & 600-900 nm PBM |
| Impact Resistance | Comparable to Gorilla Glass/Dragontrail ™ |
| Scratch Resistance | ≥9H Hardness |

Another example embodiment comprises wavelength conversion materials usable within the disclosed glass, film, or coating structures may include but are not limited to:

### Quantum Dots

CdSe, CdS, CdTe, CdZnS,
InP, InAs,
ZnSe, ZnS, ZnTe,
CuInS₂, CuInSe₂,
perovskite quantum dots,
carbon quantum dots,
graphene quantum dots,
core-shell engineered nanocrystals.

### Phosphors

YAG:Ce,
SrAl₂O₄:Eu,
Mn⁴⁺ doped fluoride phosphors,
silicate, nitride, oxynitride, or aluminate phosphors,
rare-earth activated phosphors.

### Organic or Hybrid Emitters

fluorescent dyes,
organometallic emitters,
photoactive pigments,
wavelength-shifting polymers.
   The conversion materials may be dispersed, laminated, embedded, coated, doped, encapsulated, or integrated into glass matrices or polymer structures.

Another example embodiment of the disclosure is to provide a multi-functional glass, film, coating or glass cover that selectively converts certain different spectral wavelengths and re-emits them from the glass or glass cover into surrounding air, environmental surfaces, or biological environments as modified beneficial spectral wavelengths that provide at least one of 405 nm or other antibacterial wavelengths and/or >600 nm photobiomodulating (PBM) spectral emissions as described in the below outline entitled “Example: Technical Specification for Antibacterial Down-Converting Glass 1.D”. The glass, film, coating or glass cover may be configured to comprise but not be limited to the below specifications and composition as outlined below in items 1-2 entitled under “Example: Technical Specification for Antibacterial Down-Converting Glass 1.D”.

### Example: Technical Specification for Antibacterial Down-Converting Glass 1.D

### 1. Description

A wavelength-selective, antimicrobial glass designed to convert ambient solar UV and blue light into 405 nm antimicrobial emission while maintaining high optical clarity. The glass integrates low-emissivity (Low-E) coatings for energy efficiency and is suitable for architectural, automotive, medical, and public infrastructure applications.

### Composition

### 2.1 Base Composition

Substrate: Low-iron float glass, laminated glass, polycarbonate, or specialty transparent materials.
Thickness Options: 2 mm-12 mm.
Visible Light Transmission: >85%.

### 2.2 Functional Additives

UV & Blue Light Absorption: Titanium Dioxide (TiO₂), Zinc Oxide (ZnO), or Nano-Structured UV Blockers.
Down-Conversion Materials: Rare-earth phosphors (YAG:Ce, SrAl₂O₄:Eu) or Quantum Dot Materials (Perovskite-Based, Encapsulated Nano-QDs).
Low-E & Infrared Control: Silver, Indium-Tin-Oxide (ITO), or Selective Metallic Coatings.

### Performance & Optical Properties

Antimicrobial Emission: 405 nm light emission via passive down-conversion.
Heat & UV Resistance: UV-stable coatings prevent degradation.
Infrared Reflectivity: Up to 85% for enhanced thermal control.

### Applications

Architectural Windows
Hospital & Cleanroom Glass
Public Transportation Screens
High-Touch Surfaces (Kiosks, Smart Glass)

Another example embodiment of the disclosure is to provide a multi-functional glass, film, coating or glass cover that selectively converts certain different spectral wavelengths and re-emits them from the glass or glass cover into surrounding air, environmental surfaces, or biological environments as modified beneficial spectral wavelengths that provide at least one of 405 nm or other antibacterial wavelengths and/or >600 nm photobiomodulating (PBM) spectral emissions as described in the below outline entitled “Example: Technical Specification for Antibacterial Display Coatings 1.E”. The glass, film, coating or glass cover may be configured to comprise but not be limited to the below specifications and composition as outlined below in items 1-2 entitled under “Example: Technical Specification for Antibacterial Display Coatings 1.E”.

### Example: Technical Specification for Antibacterial Display Coatings 1.E

### 1. Description

A wavelength-selective display coating designed for LCD, OLED, and edge-lit displays to passively convert blue light (~450 nm) into antimicrobial 405 nm light, ensuring continuous self-sterilization without affecting display color accuracy.

### 2. Composition

### 2.1 Base Composition

Substrate Compatibility: Glass, polycarbonate, or polymer-based display covers.
Optically Transparent Binder: Silane-Based Dispersions, Sol-Gel Coatings, or Polyurethane-Based Polymers.

### 2.2 Functional Additives

405 nm Conversion Material: Rare-Earth Phosphors (YAG:Ce, SrAl₂O₄:Eu) or Perovskite-Based Quantum Dots.
Heat Stabilizers: Encapsulation Coatings for LED Resistance.

### Performance & Optical Properties

Light Transmission: ≥90% in non-converted areas.
Coating Methods: Spray-coating, dip-coating, or integration into display layers.
Durability: Resistant to LED heat emissions.

### Applications

Smartphones, Tablets, & Monitors
Medical Displays & Public Screens
Strengthened Glass For Display Covers
Television Screens (Room-Wide Disinfection)

Another example embodiment of the disclosure is to provide a multi-functional glass, film, coating or glass cover that selectively converts certain different spectral wavelengths and re-emits them from the glass or glass cover into surrounding air, environmental surfaces, or biological environments as modified beneficial spectral wavelengths that provide at least one of 405 nm or other antibacterial wavelengths and/or >600 nm photobiomodulating (PBM) spectral emissions as described in the below outline entitled “Example: Technical Specification for Selective Red/IR Emission Displays 1.F”. The glass, film, coating or glass cover may be configured to comprise but not be limited to the below specifications and composition as outlined below in items 1-2 entitled under “Example: Technical Specification for Selective Red/IR Emission Displays 1.F”.

### Example: Technical Specification for Selective Red/IR Emission Displays 1.F

### 1. Description

A wavelength-converting display enhancement that enables a dedicated region of a screen to convert blue backlighting into 670 nm red light or near-infrared (>700 nm) for eye health, biometric authentication, AR enhancements, or military applications.

### Composition

### 2.1 Base Composition

Display Substrate: Compatible with LCD, OLED, and Edge-Lit Screens.
Selective Quantum Dot or Phosphor Layer: Integrated into backlight unit, edge-lit guide, or screen surface.
Micro-Optics Integration: Optional micro-lenses to optimize beam shaping.

### 2.2 Functional Additives

670 nm & IR Emission Phosphors: Rare-Earth-Based Quantum Dots or Encapsulated Perovskite-Based Down-Converters.
Heat & Light Stabilization: Encapsulation for spectral consistency.

### Performance & Optical Properties

Dedicated Region Emission: 0.5″-3″ strip in the display for red/IR.
Minimal Impact to Display Clarity: Maintains transparency in non-converted areas.
Light Source Compatibility: Works with LCD backlighting, edge-lit panels, and OLED-based solutions.

### Applications

Biometric Face Recognition
Night Vision & Tactical Displays
Augmented Reality Enhancements
Photobiomodulation for Eye Health

Another example embodiment of the disclosure is to provide a multi-functional glass, film, coating or glass cover comprising high transparency with phosphor and/or quantum dot integration that absorbs one or more spectral wavelengths and converts and re-emits the illumination at different wavelengths from the glass or glass, film, coating and/or glass cover into surrounding air, environmental surfaces, or biological environments as modified beneficial spectral wavelengths that provide at least one of 405 nm or other antibacterial wavelengths and/or >600 nm photobiomodulating (PBM) spectral emissions as described in the below outline entitled “Example: Detailed Description of Transparency and Phosphor/Quantum Dot Integration 1.G”. The glass, film, coating or glass cover may be configured to comprise but not be limited to the below specifications and composition as outlined below under “Example: Detailed Description of Transparency and Phosphor/Quantum Dot Integration 1.G”.

### Example: Detailed Description of Transparency and Phosphor/Quantum Dot Integration 1.G

One of the key challenges in integrating phosphors or quantum dots into wavelength-selective glass is maintaining high optical transparency while ensuring efficient spectral conversion. This disclosed structure overcomes these challenges by including a glass, film, coating or glass cover configured to comprise and/or be capable of:
Nano-Optimized Phosphor Integration—The use of nano-sized phosphor particles (<100 nm) ensures that the glass remains highly transparent while performing spectral conversion. Larger phosphor particles (>500 nm) would scatter visible light and reduce clarity, making them unsuitable for transparent applications.
Use of Transparent Quantum Dots—Instead of traditional yellowish phosphors, transparent perovskite quantum dots or low-scattering phosphors can be embedded in the glass laminate, polymer matrix, or coating without significantly impacting transparency.
Laminated Structure for Optical Clarity—In one embodiment, the phosphor or quantum-dot layer is encapsulated within a laminated interlayer, ensuring durability while preserving at least 90-98% visible light transmission.
Spectral Filtering to Maintain Visual Appearance—The wavelength-selective materials are designed to only absorb UV and blue light (400-490 nm) while allowing the majority of visible light to pass through unaltered.
Matching Refractive Indices to Prevent Haze—By embedding phosphors in a refractive-index-matched polymer or glass interlayer, internal reflections and haze are minimized, ensuring aesthetically clear glass.
Selective Layering for Maximum Transparency—
   In architectural glass, a thin laminated interlayer is used to allow full functionality without impacting glass aesthetics.
   In display glass, phosphors or quantum dots are applied as a nano-thin optical layer optimized for LCD and OLED emissions.
      With these approaches, the glass maintains a clear, window-like appearance while effectively converting harmful UV/blue light into beneficial 405 nm, 600-700 nm, and >700 nm emissions.

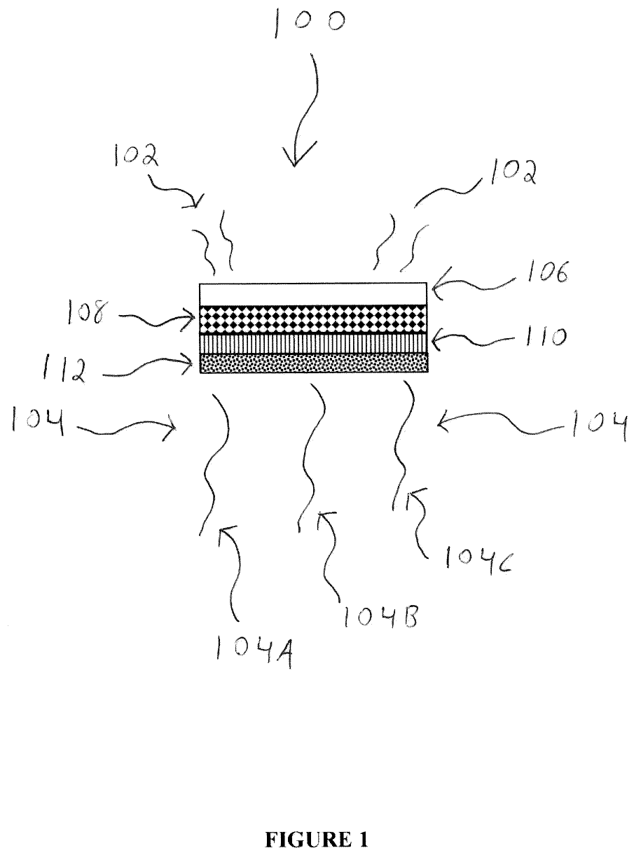

FIG. 1 shows and describes one example embodiment according to the disclosure comprising a wavelength-selective glass 100 configured to be a single layer of glass that passively converts wavelengths of incoming light 102 such as Sunlight and/or Artificial Light including but not limited to UV, white and/or blue light, and re-emits those original wavelengths of incoming light 102 into different beneficial wavelengths of light 104 including but not limited to emissions of 405 nm antimicrobial light 104A, emissions of 600 nm-700 nm photobiomodulation (PBM) Red light 104B and/or emissions of >700 nm PBM Infrared (IR) light 104C out form the other side of the wavelength-selective glass 100. The sources of artificial light providing the wavelengths of incoming light 102 may include but not be limited to any artificial light sources including but not limited to room lights, street lights, head lights, display lights including but not limited to display backlights, micro-LEDs in displays and/or OLED displays. The wavelength-selective glass 100 is configured to comprise a base glass substrate 106 which may be a transparent glass substrate with capacitive touch functionality configured to comprise a wavelength selective or conversion layer 108 and or integrated material comprising at least one wavelength conversion material such as including but not limited to rare-earth phosphors or quantum dots, that absorb specific portions of the visible and ultraviolet spectrum of received light 102 and convert at least some of or a substantial portion of the received light 102 into at least one or more of the beneficial wavelengths of light 104 and re-emit the beneficial wavelengths of light 104 including but not limited to at least one of 104A, 104B and/or 104C from the wavelength-selective glass 100 glass. The wavelength-selective glass 100 may be configured to optionally comprise a reflective layer 110 to enhance light emission efficiency by directing more of the converted beneficial wavelengths of light 104 outward from the glass. The wavelength-selective glass 100 may further comprise a coated interlayer within the glass structure that ensures long-term spectral conversion, making it ideal for building facades, hospital partitions, vehicle windows, and self-sterilizing surfaces. The wavelength-selective glass 100 may further be configured to comprise an anti-reflective coating 112 configured to improve visibility and reduce glare. The wavelength-selective glass 100 may further be configured to comprise integration with Low-E coatings to maintain thermal efficiency while providing active spectral conversion. The wavelength-selective glass 100 is particularly suited for public infrastructure, healthcare environments, transportation, smart windows, eyewear, video display devices including but not limited to wearable displays where microbial control and light-based health benefits enhance safety and comfort. The 405 nm antimicrobial wavelengths of light 104A emitting from the glass would be disinfecting the air and/or surfaces without needing any power from the glass. The >600 nm PBM wavelengths of light 104B and/or 104C emitting from the glass would be beneficial to overall cellular health including but not limited to improving vision with 670 nm emissions from the glass in some applications and not needing any power from the glass.

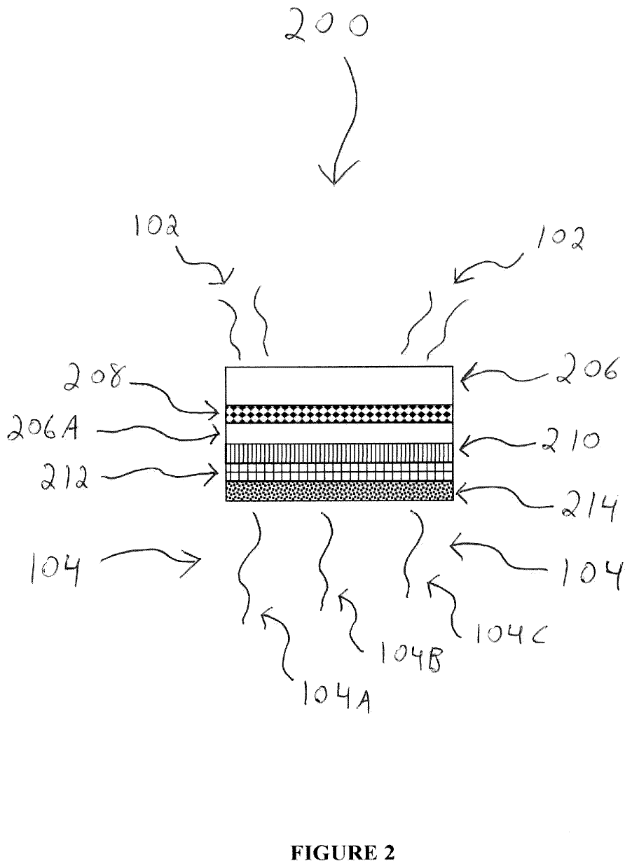

FIG. 2 shows and describes another example embodiment according to the disclosure in which a wavelength-selective glass 200 is provided similar to the single layer wavelength-selective glass 100 described in FIG. 1, but in a laminated and/or multi-layer glass embodiment. The wavelength-selective glass 200 is configured to passively convert incoming light 102 such as Sunlight and/or Artificial Light including but not limited to UV, white and/or blue light, and re-emits those original wavelengths of light 102 into beneficial wavelengths of light 104 including but not limited to emissions of 405 nm antimicrobial light 104A, emissions of 600 nm-700 nm photobiomodulation (PBM) Red light 104B and/or emissions of >700 nm PBM Infrared (IR) light 104C form the wavelength-selective glass 200. The sources of artificial light providing the wavelengths of incoming light 102 may include but not be limited to any artificial light sources including but not limited to room lights, street lights, head lights, display lights including but not limited to display backlights, micro-LEDs in displays and/or OLED displays. The wavelength-selective glass 200 is configured to comprise a base glass substrate 206 which may be a transparent glass substrate with capacitive touch functionality configured to comprise a wavelength selective or conversion layer 208 and/or integrated material comprising at least one wavelength conversion material such as including but not limited to rare-earth phosphors or quantum dots, that absorb specific portions of the visible and ultraviolet spectrum from received light 102 and convert at least some of or a substantial portion of the received light 102 into at least one or more of the beneficial wavelengths of light 104 and re-emit the beneficial wavelengths of light 104 including but not limited to at least one of 104A, 104B and/or 104C from the wavelength-selective glass 200. The wavelength-selective glass 200 may be configured to optionally comprise a reflective layer 210 to enhance light emission efficiency by directing more of the converted beneficial wavelengths of light 104 outward from the wavelength-selective glass 200. The wavelength-selective glass 200 may be configured to comprise an additional laminated layer of glass 206A for safety glass applications that require more strength. The wavelength-selective glass 200 may further comprise a coated interlayer 212 within the glass structure that ensures long-term spectral conversion, making it ideal for building facades, hospital partitions, vehicle windows, and self-sterilizing surfaces. The wavelength-selective glass 200 may further be configured to comprise an anti-reflective coating 214 configured to improve visibility and reduce glare. The wavelength-selective glass 200 may further be configured to comprise integration with Low-E coatings to maintain thermal efficiency while providing active spectral conversion. The wavelength-selective glass 200 is particularly suited for public infrastructure, healthcare environments, transportation, and smart windows, where microbial control and light-based health benefits enhance safety and comfort. The 405 nm antimicrobial wavelengths of light 104A emitting from the glass would be disinfecting the air and/or surfaces without needing any power from the glass. The >600 nm PBM wavelengths of light 104B and/or 104C emitting from the glass would be beneficial to overall cellular health including but not limited to improving vision with 670 nm emissions from the glass in some applications and not needing any power from the glass.

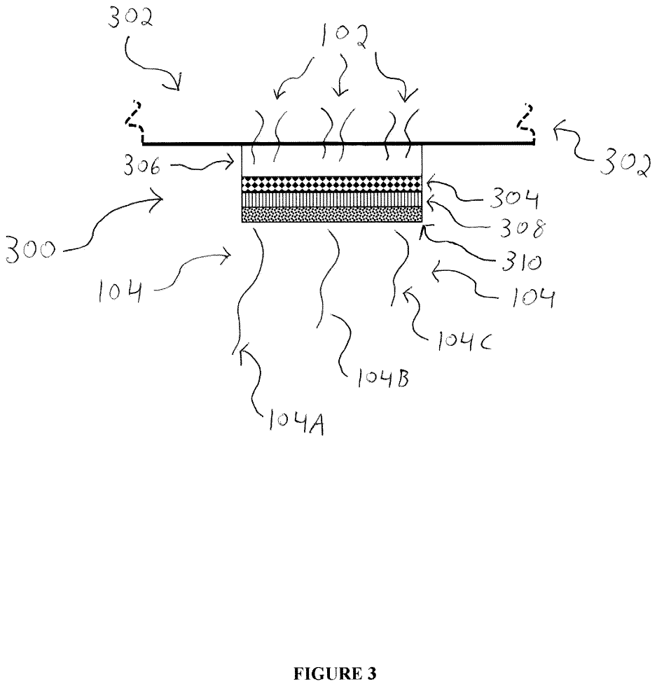

FIG. 3 shows and describes another example embodiment according to the disclosure in which a wavelength-selective display glass 300 is provided for a video display screen in a video display device 302 including but not limited to LCD screens. The wavelength-selective display glass 300 is configured to passively convert incoming light 102 from the artificial LED backlight emission from LCD pixels of the video display device 302 into beneficial wavelengths of light 104 emissions and re-emit those original wavelengths of incoming light 102 into beneficial wavelengths of light 104 including but not limited to emissions f 405 nm antimicrobial light 104A, emissions of 600 nm-700 nm photobiomodulation (PBM) Red light 104B and/or emissions of >700 nm PBM Infrared (IR) light 104C from the wavelength-selective display glass 300 that is integrated in the video display device 302, while still maintaining color accuracy and display clarity. The wavelength-selective display glass 300 is configured to comprise a coated or embedded wavelength-selective layer 304 applied to and/or integrated with the glass substrate 306, or applied to the cover glass or integrated within the LCD light guide panel (LGP) of the video display device 302. The embedded wavelength-selective layer 304 is configured to include a material such as phosphors, quantum dots or other spectral conversion materials for Selective Absorption of blue wavelengths (400-470 nm) and re-emission at 405 nm (antimicrobial) and/or red/IR PBM light (>600 nm) from the wavelength-selective display glass 300 and/or video display device 302. The wavelength-selective display glass 300 may be configured to optionally comprise a reflective layer 308 to enhance light emission efficiency by directing more of the converted beneficial wavelengths of light 104 outward from the wavelength-selective display glass 300. The wavelength-selective display glass 300 may further be configured to comprise an anti-reflective coating 310 configured to improve visibility and reduce glare. The video display device 302 may be configured to comprise hardware, software and/or AI capabilities configured for Optimization for LCD backlighting configurations, allowing for minimal power consumption while improving display comfort and health benefits. The wavelength-selective display glass 300 and/or video display device 302 may be configured to be designed for consumer electronics, medical displays, and large-format commercial screens where viewing and/or extended viewing requires eye protection, improved overall cellular health and integrated antimicrobial functionality. The wavelength-selective display glass 300 may be configured to be applied to any display system utilizing a glass substrate, including but not limited to LCDs, OLEDs, micro-LEDs, and other emissive or transmissive display technologies including but not limited to wearable augmented reality (AR), virtual reality (VR) displays and/or AR/VR optics, designed to enhance near-eye comfort and health benefits while maintaining high optical clarity. The wavelength-selective layer functions by absorbing incident blue light, whether generated from a backlight or self-emissive pixels, and converting a portion into 405 nm antimicrobial and/or >600 nm PBM wavelengths.

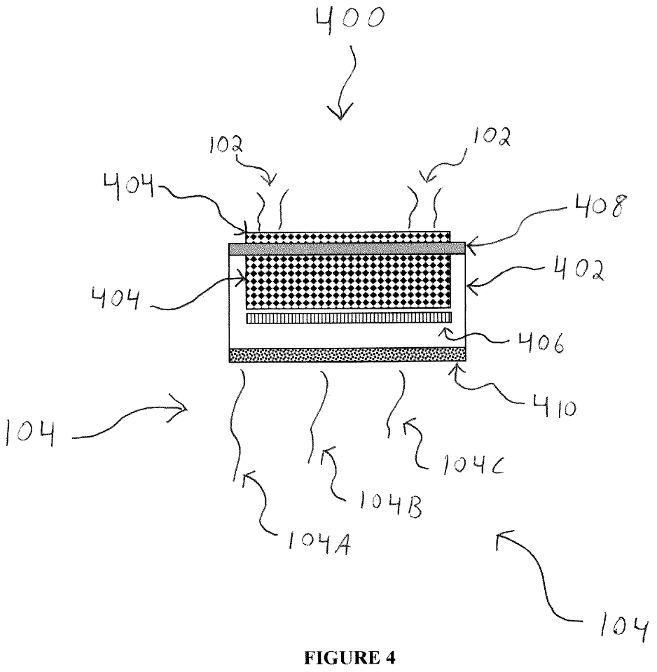

FIG. 4 shows and describes another example embodiment according to the disclosure being configured to provide a strengthened and/or long-term wavelength-selective glass 400 via infused or laminated structures of an outer glass layer or glass matrix 402, ensuring durability, optical stability, and high-performance spectral conversion. The wavelength-selective glass 400 is configured to passively convert incoming light 102 from the sun and/or artificial light into beneficial wavelengths of light 104 emissions and re-emit those original wavelengths of incoming light 102 into beneficial wavelengths of light 104 including but not limited to emissions of 405 nm antimicrobial light 104A, emissions of 600 nm-700 nm photobiomodulation (PBM) Red light 104B and/or emissions of >700 nm PBM Infrared (IR) light 104C from the wavelength-selective glass. The wavelength-selective glass 400 may be configured to comprise a phosphor-embedded laminated interlayer 404 between two or more glass layers to provide long-lasting spectral conversion. The phosphor-embedded laminated interlayer 404 comprises encapsulation of quantum dots or down-converting phosphors within the glass matrix to ensure resistance to environmental degradation. The wavelength-selective glass 400 is configured for high-temperature, high-pressure compatibility, making it suitable for aviation, space, and defense applications where durability is critical. The wavelength-selective glass 400 may be configured to optionally comprise a reflective layer 406 to enhance light emission efficiency by directing more of the converted beneficial wavelengths of light 104 outward from the wavelength-selective glass 400. The wavelength-selective glass 400 is configured to comprise a laminated safety layer 408 for structural integrity and resistance to shattering. The wavelength-selective glass 400 may further be configured to comprise an anti-reflective coating 410 configured to improve visibility and reduce glare.

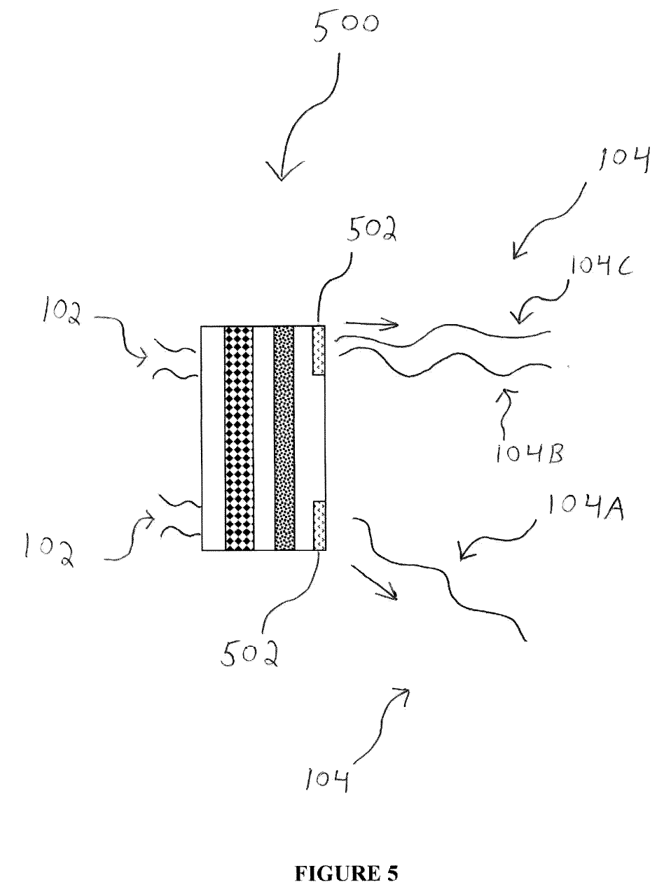

FIG. 5 shows and describes another example embodiment according to the disclosure being configured to comprise a wavelength-selective glass 500, which may be configured to be any one of the embodiments as described in FIGS. 1-4. The wavelength-selective glass 500 is also configured to passively convert wavelengths of incoming light 102 such as Sunlight and/or Artificial Light including but not limited to UV, white and/or blue light, and re-emit those original wavelengths of incoming light 102 into different beneficial wavelengths of light 104 including but not limited to emissions of 405 nm antimicrobial light 104A, emissions of 600 nm-700 nm photobiomodulation (PBM) Red light 104B and/or emissions of >700 nm PBM Infrared (IR) light 104C out form the other side of the wavelength-selective glass 500. However in this embodiment the wavelength-selective glass 500 is configured to comprise and/or emit the beneficial wavelengths of light 104 through micro-optics 502. The micro-optics may be configured to be an integrated part of the wavelength-selective glass 500 or placed over it and may further be configured to be integrated and/or placed over the entire wavelength-selective glass 500 or integrated or placed over at least one select region(s) of the wavelength-selective glass 500. The micro-optics 502 may be configured to enable the wavelength-selective glass 500 to emit the beneficial wavelengths of light 104 in different emission beam angles and or emit only one type of particular beneficial wavelength from one region such antimicrobial light 104A in a downward direction towards a surface to provide anti-microbial light emission onto the surface such as a table or a keyboard while also being configured to emit the PBM wavelengths of light 104B and/or 104C towards a different direction such as towards a person looking thought a window or at a video display device (including but not limited to a wearable display device) configured to comprise and one of the wavelength selective glass embodiments according to the disclosure provided herein.

In some embodiments, the wavelength-selective glass, film and/or coating systems may operate continuously in response to environmental illumination without user intervention.

### Example Methods Include:

converting sunlight entering a building into antimicrobial and photobiomodulation emissions within interior spaces;
converting display emissions into biologically beneficial wavelengths directed toward a user;
converting vehicle window sunlight into therapeutic emissions directed toward occupants;
providing simultaneous antimicrobial environmental treatment and photobiomodulation exposure.
   The systems may function under varying illumination intensities and across indoor or outdoor environments.

The various structural features, material compositions, wavelength ranges, emission characteristics, encapsulation approaches, optical management structures, and application environments described herein may be implemented individually or in any combination or sub-combination, and the disclosure expressly contemplates embodiments directed to any such combinations and sub-combinations, whether or not explicitly illustrated or described in a single embodiment

As used herein, the term “biological environment” refers to any environment in which one or more biological entities are present, including but not limited to humans, animals, plants, microorganisms, agricultural systems, food storage systems, and other living or biologically active systems.

While the foregoing there has set forth embodiments of the disclosure, it is to be understood that the present disclosure may be embodied in other forms without departing from the spirit or central characteristics thereof. The present embodiments, therefore, are to be considered in all respects as illustrative and not restrictive, and the disclosure is not to be limited to the details given herein. While specific embodiments have been illustrated and described, numerous modifications come to mind without significantly departing from the characteristics of the disclosure and the scope of protection is only limited by the scope of the accompanying claims.

The invention is claimed as follows:

1. A wavelength-converting optical film comprising:
at least one layer comprising a wavelength-converting material configured to absorb at least a first wavelength range of ambient light and emit at least a second wavelength range different from the first wavelength range,
wherein the wavelength-converting optical film is configured for placement on or within a non-wearable environmental structure and, upon exposure to ambient light, to provide passive antimicrobial emission of light.

2. The wavelength-converting optical film of claim 1, wherein the second wavelength range comprises approximately 350 nm to 450 nm.

3. The wavelength-converting optical film of claim 1, wherein the wavelength-converting material is positioned between at least two protective barrier layers.

4. The wavelength-converting optical film of claim 3, wherein the at least two protective barrier layers comprise polymer films configured to reduce oxygen and moisture ingress.

5. The wavelength-converting optical film of claim 1, wherein the wavelength-converting material is dispersed within a polymer matrix.

6. The wavelength-converting optical film of claim 1, wherein the wavelength-converting optical film is configured for attachment to a glass substrate, a glazing unit, or a transparent panel.

7. The wavelength-converting optical film of claim 1, further comprising at least one optical structure to modify distribution or direction of emitted light, the at least one optical structure selected from micro-optics, diffusers, waveguides, light-guide layers, patterned emission regions, reflective layers, and refractive-index-modifying layers configured.

8. The wavelength-converting optical film of claim 1, wherein the wavelength-converting optical film is configured to maintain visible light transmission of at least 80 percent.

9. A wavelength-converting optical film comprising:
at least one layer comprising a wavelength-converting material configured to absorb at least a first wavelength range of ambient light and emit at least a second wavelength range different from the first wavelength range,
wherein the wavelength-converting optical film is configured for placement on or within a non-wearable environmental structure to modify ambient illumination within a biological environment and, upon exposure to ambient light, to provide passive photobiomodulation emission of light.

10. The wavelength-converting optical film of claim 9, wherein the second wavelength range comprises approximately 600 nm to 1 millimeter.

11. The wavelength-converting optical film of claim 9, wherein the wavelength-converting material is incorporated within a multilayer film stack.

12. The wavelength-converting optical film of claim 9, wherein the wavelength-converting material comprises quantum dots, phosphors, luminescent dyes, rare-earth materials, or combinations thereof.

13. The wavelength-converting optical film of claim 9, wherein the biological environment includes at least one of plants, animals, microorganisms, or agricultural systems.

14. The wavelength-converting optical film of claim 9, wherein the wavelength-converting optical film is configured for greenhouse glazing, livestock enclosures, aquaculture environments, or food processing environments.

15. The wavelength-converting optical film of claim 9, wherein the emitted light is preferentially directed through one major surface of the wavelength-converting optical film.

16. The wavelength-converting optical film of claim 9, wherein the wavelength-converting material is configured to emit multiple photobiomodulation wavelengths.

17. A wavelength-converting glass structure comprising:
a glass substrate; and
at least one wavelength-converting material incorporated within, coated on, laminated within, or doped into the glass substrate,
wherein the wavelength-converting glass structure is configured to absorb ambient light and emit converted light through at least one surface of the glass substrate into a surrounding environment to modify ambient illumination.

18. The wavelength-converting glass structure of claim 17, wherein the at least one wavelength-converting material is positioned within a laminated interlayer between at least two glass layers.

19. The wavelength-converting glass structure of claim 17, further comprising a low-emissivity coating.

20. The wavelength-converting glass structure of claim 17, wherein the glass substrate comprises architectural glass, vehicle glass, or display cover glass.

21. The wavelength-converting glass structure of claim 17, further comprising at least one optical management structure configured to direct emitted light toward an environment adjacent the wavelength-converting glass structure.

22. A wavelength-modifying environmental system comprising:
at least one transparent structure configured to receive ambient light; and
at least one wavelength-converting material associated with the at least one transparent structure and configured to convert ambient light into biologically beneficial emissions of light without dedicated electrical power.

23. The wavelength-modifying environmental system of claim 22, wherein the at least one transparent structure comprises architectural glazing.

24. The wavelength-modifying environmental system of claim 22, wherein the at least one transparent structure comprises a wavelength-converting optical film.

25. The wavelength-modifying environmental system of claim 22, wherein the biologically beneficial emissions of light include antimicrobial wavelengths.

26. The wavelength-modifying environmental system of claim 22, wherein the biologically beneficial emissions of light include photobiomodulation wavelengths.

27. The wavelength-modifying environmental system of claim 22, wherein the wavelength-modifying environmental system operates under natural sunlight conditions.

28. The wavelength-modifying environmental system of claim 22, wherein the wavelength-modifying environmental system operates under artificial indoor illumination.

29. The wavelength-modifying environmental system of claim 22, wherein the emitted light modifies environmental illumination within a human-occupied or biological environment.

30. The wavelength-modifying environmental system of claim 22, wherein the at least one wavelength-converting material is configured to operate across varying illumination intensities.
